(19) United States
(12) Patent Application Publication
(10) Pub. No.: US 2020/0129779 A1
(43) Pub. Date: Apr. 30, 2020

(54) IMPLANT, DIAGNOSIS AND TREATMENT DEVICE, AND METHOD OF EMITTING LASER

(71) Applicants: BEIJING BOE DISPLAY TECHNOLOGY CO., LTD., BEIJING (CN); BOE TECHNOLOGY GROUP CO., LTD., BEIJING (CN)

(72) Inventors: Xin He, Beijing (CN); Jiliang Zhang, Beijing (CN); Enqiang Zheng, Beijing (CN); Xiaopeng Cui, Beijing (CN)

(21) Appl. No.: 16/549,420

(22) Filed: Aug. 23, 2019

(30) Foreign Application Priority Data
Oct. 31, 2018 (CN) 201811287926.8

(51) Int. Cl. A61N 5/06; A61L 31/16; A61L 27/54; A61L 31/10

(52) U.S. Cl. CPC A61N 5/062; A61L 31/16; A61L 31/022; A61L 31/10; A61L 27/54

(57) ABSTRACT

An implant for laser diagnosis and treatment comprises: a metal halide perovskite, wherein the metal halide perovskite is in a form of a nanosheet, a nanowire, or a quantum dot; a gold nanoshell coupled to the metal halide perovskite; and an antibody, which is bondable to a biological tissue, on an outer surface of the gold nanoshell. A diagnosis and treatment device for exciting the implant, a method of using the implant, and a diagnosis and treatment system are also disclosed.

### CROSS-REFERENCE TO RELATED APPLICATION

This application claims the priority of Chinese Patent Application No. 201811287926.8 filed on Oct. 31, 2018, which is incorporated herein in its entirety by reference.

### TECHNICAL FIELD

This disclosure relates to the field of medical lasers, and particularly to an implant, a diagnosis and treatment device, and a method of emitting a laser.

### BACKGROUND

Laser technology has been a new light source since 1960s due to good directional property, high brightness, good monochromaticity, high energy density, and the like. Laser industry based on laser devices has been rapidly developed in the world. As new laser devices continuously appear and laser medical studies develop, the laser technology has been successfully used in clinical practice since 1970s. At present, laser medical applications have permeated into various subjects such as ophthalmology, dermatology, cardiovasology, and the like.

At present, commonly used medical laser sources include an argon ion laser source, a diode laser source, a CO2 laser source, and the like. Medical laser sources in the prior art are all in vitro laser sources. In vitro light sources emit laser acting on tissues of interest. In the process of application, if the protection against laser emitted from an external light source is neglected, normal tissues of a human body will be easily damaged by laser, resulting in an irreversible damage of the human body.

Lasers having various wavelengths and energy radiations thereof will lead to irreversible damages to various tissues of a human body. For example, with respect to the damage to the eye caused by laser, excessive heat is generated when laser focuses on a photoreceptor cell, and the protein coagulation denaturation caused thereby is an irreversible damage. Once it is damaged, permanent blindness of the eye will be caused. For further example, when laser irradiates the skin, the damage to the skin may be caused if the laser has an excessively large energy (power). The mechanism of the damage to the skin caused by laser is mainly the heat effect of laser.

### SUMMARY

In one aspect, this disclosure provides an implant for laser diagnosis and treatment, wherein the implant comprises:
a metal halide perovskite, wherein the metal halide perovskite is in a form of a nanosheet, a nanowire, or a quantum dot;
a gold nanoshell coupled to the metal halide perovskite; and
an antibody, which is bondable to a biological tissue, on an outer surface of the gold nanoshell.

Optionally, the metal halide perovskite is a metal halide perovskite having a two-dimensional structure.

Optionally, the metal halide perovskite has a general chemical formula of AMX₃, wherein A represents a monovalent cation, M represents a bivalent metal ion, and X represents a halogen ion.

Optionally, A is selected from CH₃NH₃⁺, Cs⁺, and Rb⁺, M is selected from Pb²⁺ and Sn²⁺, and X is selected from Cl⁻, Br⁻, and I⁻.

Optionally, the antibody is a specific antibody.

Optionally, the specific antibody comprises a protein, a polypeptide, DNA, or a drug.

Optionally, the surface of the gold nanoshell is coupled to the antibody through a ligand.

Optionally, a therapeutic targeted drug is supported on an internal cavity or a surface of the gold nanoshell.

In another aspect, this disclosure provides a laser diagnosis and treatment device, wherein the laser diagnosis and treatment device comprises:
a light source module configured to emit visible light or infrared light for exciting the implant described above in an organism to emit laser.

Optionally, the light source module comprises a light source emitter, a first beam splitter, a power meter, and a microscope, wherein the first beam splitter splits light emitted from the light source emitter to the implant, the power meter, and the microscope.

Optionally, the diagnosis and treatment device further comprises:
an imaging module configured to image the laser emitted from the implant under excitation of the light source module.

Optionally, the imaging module comprises a second beam splitter and a plurality of imaging apparatuses, wherein the second beam splitter splits the laser to the plurality of imaging apparatuses.

Optionally, the plurality of imaging apparatuses comprise a camera and an optical coherence imager.

In yet another aspect, this disclosure provides a method of emitting laser by using the implant described above, wherein the method comprises:
allowing the implant to be bonded to a biological tissue in an organism by the antibody, and
emitting visible or near infrared light by using an external light source outside the organism to excite the implant to emit laser.

Optionally, the visible or near infrared light has an energy density between 1×10⁻⁷ J cm⁻² to 1×10⁻⁵J cm⁻².

Optionally, the biological tissue is subjected to laser treatment or laser imaging by using the laser.

### BRIEF DESCRIPTION OF THE DRAWINGS

In order to illustrate the technical solutions of this disclosure more clearly, accompanying drawings will be simply introduced below. It is apparent that the accompanying drawings described below are merely some embodiments related to this disclosure but not limitations of this disclosure.

FIG. 1 is a schematic diagram of an embodiment according to this disclosure;

FIG. 2 is a schematic diagram of wavelength ranges of lasers emitted from some metal halide perovskites after excitation;

FIG. 3 is a structural schematic diagram of surface modification of a gold nanoshell with a specific antibody in an embodiment according to this disclosure;

FIG. 4 is a structural schematic diagram of a laser diagnosis and treatment system in an embodiment according to this disclosure;

FIG. 5 is a block diagram of function realization of a laser diagnosis and treatment system in an embodiment according to this disclosure; and

FIG. 6 is a flow chart of laser diagnosis and treatment in an embodiment according to this disclosure.

### DETAILED DESCRIPTION OF EMBODIMENTS

As found by the inventor, a method of forming a medical laser source, which may act on tissues of interest in a directed manner and may prevent the damage to normal tissues of an organism caused by an emitted laser or an excitation light source of laser, as well as a laser treatment apparatus, and a laser diagnosis and treatment system are desired. The inventor provides this disclosure to solve the technical problems described above present in the prior art.

In order to enable objects, technical solutions, and advantages of embodiments of this disclosure to be clearer, technical solutions of embodiments of this disclosure will be described clearly and fully below in conjunction with accompanying drawings of embodiments of this disclosure. Obviously, the embodiments described are a part of the embodiments of this disclosure, rather than all of the embodiments. Based on the embodiments described of this disclosure, all other embodiments obtained by those of ordinary skill in the art without performing inventive work also belong to the scope protected by this disclosure.

Unless defined otherwise, technical terms or scientific terms used in this disclosure should have general meanings as understood by those of ordinary skill in the art to which this disclosure belongs. The word, such as “include”, “comprise”, or the like, used in this disclosure means that the element or article occurring before this word encompasses the element or article and the equivalent thereof enumerated after this word and does not exclude other elements or articles. The word, such as “connection”, “attachment”, or the like, is not limited to a physical or mechanical connection, but may include an electric connection, either direct or indirect. The word, such as “above”, “below”, “left”, “right”, or the like, is only used to indicate a relative position relationship. After the absolute position of a described object is changed, this relative position relationship may be changed accordingly.

In order to maintain the following descriptions of the embodiments in this disclosure to be clear and brief, detailed descriptions of known functions and known members are omitted in this disclosure.

This disclosure provides an implant for laser diagnosis and treatment, wherein the implant comprises:
a metal halide perovskite, wherein the metal halide perovskite is in a form of a nanosheet, a nanowire, or a quantum dot;
a gold nanoshell coupled to the metal halide perovskite; and
an antibody, which is bondable to a biological tissue, on an outer surface of the gold nanoshell.

In this disclosure, the metal halide perovskite refers to a material having a perovskite-type lattice structure and comprises a metal and a halogen in its composition. As well known, there are three lattice sites in a perovskite-type lattice, which may be represented as ABX₃. In the metal halide perovskite of this disclosure, a halogen is in position X, a metal is in at least one of sites A and B, and a metal is preferably in site B.

The metal halide perovskite is in a form of a nanosheet, a nanowire, or a quantum dot. Laser sources formed by metal halide perovskites in these forms have small volume, and will be easily accepted by an organism and discharged from the organism after use.

The implant of this disclosure has a gold nanoshell coupled to the metal halide perovskite. The gold nanoshell is a spherical-shell-like nanomaterial formed from gold. The gold nanoshell may be coupled to the metal halide perovskite in various manners, including but not limited to, being coupled through a ligand.

An antibody, which is bondable to a biological tissue, is present on an outer surface of the gold nanoshell. The antibody is bondable to a corresponding biological tissue of interest so that the implant comprising the metal halide perovskite is bonded to the biological tissue after the implant enters an organism. For example, the antibody may bind to an antigen in a diseased biological tissue so as to fix the implant in a disease part. The implant may be used in an organism in the manner described above.

FIG. 1 shows a schematic diagram of a method of forming an implant by using a metal halide perovskite in an embodiment according to this disclosure. As shown in FIG. 1, the method comprises: exciting an implant 14 in a state of being bonded to a tissue 15 of interest by visible and near infrared light 13 applied from the outside of an organism 11, for example from a light source 12, to emit laser 16. The implant 14 comprises a metal halide perovskite 141. A metal halide perovskite used in the field of solar cells may be used, which has high optical gain, high absorption coefficient, and low defect density. As inventively found by the inventor through clinical trials, the laser source 14 exhibits good metabolism after being ingested into the organism 11 and is also relatively safe for the organism 11, and therefore it may be used as a laser treatment formulation as described above. The implant is an in situ laser source, and the laser 16 generated thereby focuses on the tissue 15 of interest in the case where the attenuation is less so as to ensure the therapeutic effect on the tissue 15 of interest (relatively good penetrability) and reduce the side effect on peripheral tissues. Here, the visible and near infrared light 13 has a relatively low energy density, so that irreversible damages to normal tissues of the organism 11 may be prevented, and the safety is relatively high.

The metal halide perovskite may be used as a laser source. The metal halide perovskite employs at least one structure of a nanosheet, a nanowire, and a quantum dot so as to form a microscale and/or nanoscale in vivo laser source under excitation. Under excitation of laser emitted from an external light source, the metal halide perovskites having these structures serve as two roles of a “mirror” and a gaining media themselves to form an optically-resonant cavity so as to increase the intensity of laser by an optical resonance effect.

The gold nanoshell has a photothermal effect under irradiation of light and may generate heat. The gold nanoshell coupled to the metal halide perovskite may be directly subjected to the effect of the laser emitted from the metal halide perovskite to generate heat, and has a good effect of heat generation. The heat generation of the gold nanoshell may directly heat biological tissues, so as to exhibit an effect of heat treatment. Furthermore, when a drug is supported by the gold nanoshell as described below, heat generation facilitates better release of the drug. Additionally, biological tissues may be directly subjected to laser treatment with the laser emitted from the metal halide perovskite. Therefore, the implant of this disclosure may achieve laser treatment, photothermal treatment, and optionally drug treatment in terms of treatment at the same time, and has a synergistic effect and an excellent overall therapeutic effect. Furthermore, gold nanoshell is coupled to the metal halide perovskite, and may also be connected to a biological tissue by an antibody. Therefore, the implant of this disclosure facilitates implantation and fixation into an organism, and facilitates discharge from the organism after use.

The implant of this disclosure may also be used in biological imaging. The implant binding to a biological tissue may be indirectly observed by detecting the laser emitted from the metal halide perovskite. On the other hand, gold nanoshell may be used as a contrast agent in laser imaging. Furthermore, gold nanoshell may also be used in ultrasonic imaging. Therefore, the implant of this disclosure may provide the shape information of the biological tissue to which it binds in various manners so as to achieve biological imaging.

In some embodiments, the implant 14 comprises a metal halide perovskite 141 having a two-dimensional structure. The two-dimensional structure means that octahedrons [BX₆]⁴⁻ in the perovskite are isolated by lattice of A to form an octahedron layer so as to exhibit two-dimensional properties. Two-dimensional properties are advantageous to laser emission. In the octahedron layer, which is the two-dimensional structure of the metal halide perovskite 141, electrons and holes will be strongly confined in the octahedron layer and are connected with each other by the Coulomb force therebetween. The binding energy of the electron-hole pair under this strong confinement is approximately hundreds of millions of electron volts and may generate a strong light-substance interaction, so that the implant 14 is excited under the action of the visible and near infrared light 13, and generates in situ laser better. In some embodiments, the intensity of the laser generated may be adjusted by the intensity (for example, energy density and the like) of the laser emitted from the external light source 12. As the energy density of the laser emitted from the external light source becomes lower, the energy density of the laser generated by the metal halide perovskite 141 under excitation is also lower.

In some embodiments, the metal halide perovskite has a general chemical formula of AMX₃, wherein A represents a monovalent cation, M represents a bivalent metal ion, and X represents a halogen ion. Optionally, the monovalent cation may be CH₃NH₃⁺, Cs⁺, Rb⁺, and the like; the bivalent metal ion may be Pb²⁺, Sn²⁺, and the like; and the halogen ion may be Cl⁻, Br⁻, I⁻, and the like. This is not specifically limited hereby. The metal halide perovskites having these compositions will be particularly easily coupled to the gold nanoshell, and the laser emitted is suitable for use in diagnosis and treatment.

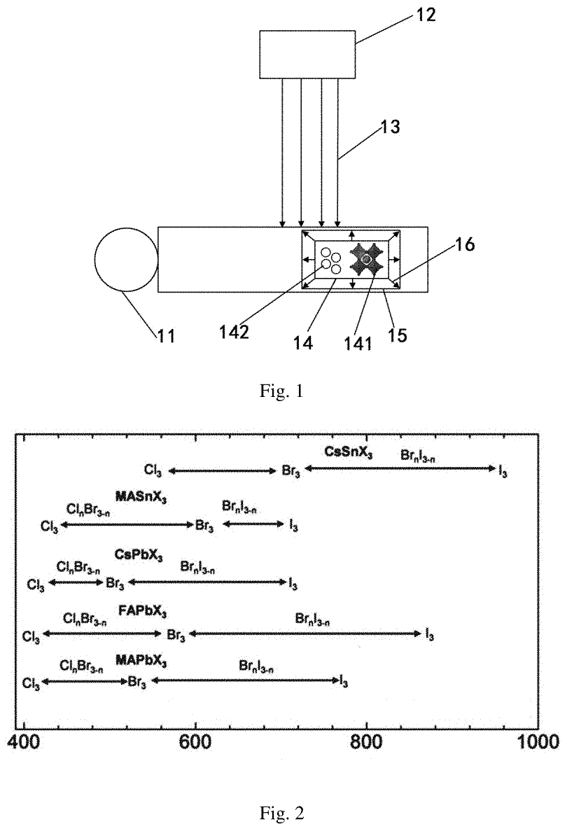

In some embodiments, the method of forming a laser source by using a metal halide perovskite 141 further comprises extending a wavelength range of laser emitted a metal halide perovskite 141 by various methods such as adjusting a stoichiometric ratio of the metal halide perovskite 141, replacing a halogen or mixed halogens, and the like, so that the laser emitted thereby has a wavelength range covering visible and near infrared regions of a desirable wavelength range or even the entire visible and near infrared regions to be adapted to various requirements in practical applications. This is not specifically limited hereby. FIG. 2 shows a schematic diagram of wavelength ranges of lasers emitted from metal halide perovskites 141 having different halogens or mixed halogens after excitation in embodiments of this disclosure. For example, as shown in FIG. 2, if the metal halide perovskite 141 in the implant 14 has a chemical formula of CsSnX₃, visible light having a wavelength of approximately 560 nm is emitted after excitation when X₃ in CsSnX₃ is Cl₃, visible and near infrared light having a wavelength of approximately 720 nm is emitted after excitation when X₃ in CsSnX₃ is Br₃, near infrared light having a wavelength of approximately 1000 nm is emitted after excitation when X₃ in CsSnX₃ is I₃, visible and near infrared light having a wavelength range of approximately 720 nm-1000 nm is allowed to be emitted after excitation when X₃ in CsSnX₃ is mixed halogens BrₙI₃₋ₙ (n≤3).

In some embodiments, the antibody is a specific antibody. In this disclosure, the antibody may be a specific antibody or a non-specific antibody. The specific antibody is particularly effective with respect to certain specific antigens. In some embodiments, the specific antibody comprises a protein, a polypeptide, DNA, or a drug.

The metal halide perovskite 141 is coupled to the gold nanoshell 142 to obtain the implant 14 (as shown in FIG. 1). The gold nanoshell may be attached to the surface of the metal halide perovskite. Here, the metal halide perovskite 141 exhibits convenient synthesis and ligand exchange as well as a simple process and will be easily combined with other nanomaterials, and may synergistically act on the tissue 15 of interest. The gold nanoshell 142 has a strong absorption in visible-near infrared regions. Therefore, the gold nanoshell 142 may respond to a window of near infrared laser for biological imaging so as to be used for imaging the tissue 15 of interest as a good biological imaging contrast agent. Furthermore, after the gold nanoshell 142 absorbs the energy of light, electrons transit from the ground state to the excited state and then transit from the excited state back to the ground state. In the process of transition back to the ground state, energy is released and transferred in a form of heat, so that the temperatures of the gold nanoshell 142 and its ambient environment are increased. Energy is transferred between the surface electron and the lattice of the gold nanoshell 142 through electron-phonon interaction and heat is transferred to the ambient environment through the lattice by phonon-phonon interaction (100-380 ps), so that the temperature of the gold nanoshell 142 itself is decreased and the temperature of the ambient environment is increased. This characteristic of the gold nanoshell 142 allows that it may be used to perform heat treatment on the tissue 15 of interest in the organism 11. Since a tumor cell has a low heat resistance compared to a normal healthy cell, it is possible to preferentially loosen tumor cell membranes and damage proteins therein by heating at an excessively high temperature, so that tumor cells are killed. In this disclosure, the gold nanoshell is coupled to the metal halide perovskite, and the energy of the laser emitted may be directly absorbed and is converted to thermal energy. Furthermore, gold nanoshell may also be used in ultrasonic imaging. A conventional gold nanoshell in the fields of medical imaging and heat treatment may be used.

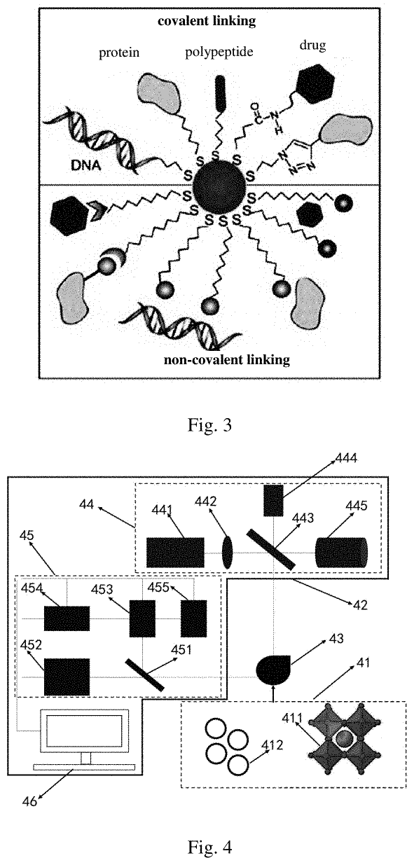

In some embodiments, the gold nanoshell 142 is coupled to an antibody. The coupling to a specific antibody or a non-specific antibody and the coupling to and/or the supporting of a therapeutic targeted drug may be achieved by surface modification. FIG. 3 is a structural schematic diagram of surface modification of a gold nanoshell with a specific antibody in an embodiment of this disclosure. As shown in FIG. 3, the specific antibody performs surface modification on the gold nanoshell 142 in a manner of covalent linking and/or non-covalent linking to improve the dispersibility, the biocompatibility, the ability of target recognition, and the like of the gold nanoshell 142. Specifically, the specific antibody includes various ones, such as proteins, polypeptides, drugs, DNA, and the like. This is not specifically limited hereby. The implant may bind to a biological tissue by means of these antibodies. In some embodiments, the coupling is achieved by ligand modification. Specifically, an amino group, a mercapto group, a carboxyl group, a phosphoric acid group, and the like each has a lone electron pair and may generate covalent interaction with gold, so that the coupling of the gold nanoshell to the specific or non-specific antibody may be achieved by ligand modification.

In some embodiments, the internal cavity and/or the surface of the gold nanoshell 142 are configured to support a drug. The gold nanoshell 142 has an internal cavity and a relatively large surface area. This structural characteristic enables the gold nanoshell 142 to support a drug. By taking full advantage of the unique structure, such as the internal cavity and the large surface area, of the gold nanoshell 142, the synthesis of multi-function particles (for example, but not limited to, supporting various drugs having various functions) is made possible.

In some embodiments, the method further comprises: controlling the radiation of the visible and near infrared light 13 applied from the outside of the organism 11 to control the release of the drug supported. Specifically, the drug supported on the surface of the gold nanoshell 142 may be released to act on the tissue 15 of interest, while the drug in the internal cavity of the gold nanoshell 142 is released by the radiation of the visible and near infrared light 13 to act on the tissue 15 of interest. By supporting drugs in the internal cavity and on the surface of the gold nanoshell and controlling the release of specific drugs through controlling externally applied radiation, the synergistic effect of photothermal treatment and drug treatment may be promoted, so as to improve the efficiency of treatment, and the biological toxicity of conventional anticancer drugs may also be decreased at the same time.

An embodiment of this disclosure further provides a laser diagnosis and treatment apparatus 42. As shown in FIG. 4, the laser diagnosis and treatment apparatus 42 is operated in cooperation with an implant 41 ingested to an organism by using the method in various embodiments according to this disclosure. The laser diagnosis and treatment apparatus 42 comprises an external light source module 44. The external light source module 44 is used to apply visible and near infrared light from the outside of an organism to excite the implant 41 in a state of being bonded to a tissue 43 of interest so as to form an in vivo laser source and emit in situ laser acting on the tissue 43 of interest, while the gold nanoshell is allowed to generate heat, so as to treat the tissue 43 of interest. Here, the energy density of the visible and near infrared light applied by the external light source module 44 is relatively low, so that normal tissues of the organism will not be damaged in the process of laser treatment performed on the tissue 43 of interest in the organism by this laser treatment apparatus 42.

In some embodiments, the visible and near infrared light applied by the external light source module 44 has an energy density in orders of magnitude of 10⁻⁷ J cm⁻² to 10⁻⁶ J cm ⁻². Optionally, the intensity of the laser emitted from the external light source module 44 is adjustable, so that the laser source formed after the metal halide perovskite 41 is excited may emit lasers having different energy densities to satisfy different requirements of treatment.

In some embodiments, the laser treatment apparatus 42 further comprises an imaging module 45 and a console 46. The imaging module 45 is used for imaging a tissue 43 of interest, the console 46 is used for receiving image data sent by the imaging module 45 and performing operations such as storing, processing, and the like so as to guide the process of treatment and to assess the therapeutic effect.

In some embodiments, the external light source module 44 comprises a light source emitter 441, an adapter 442, a first beam splitter 443, a power meter 444, and a microscope 445, wherein the adapter 442 is used for adapting the first beam splitter 443, the visible and near infrared light emitted from the light source emitter 441 is transmitted to the first beam splitter 443 and is then transmitted through the first beam splitter 443 to each of the tissue 43 of interest, the power meter 444, and the microscope 445.

In some embodiments, the imaging module 45 comprises a second beam splitter 451, a camera 452, an optical coherence imager 453, a confocal scanning microscope 454, and a photoacoustic imager 455, wherein the laser emitted the in vivo laser source is transmitted to the second beam splitter 451 and is then transmitted through the second beam splitter 451 to each of the plurality of imaging apparatuses. The imaging apparatus may comprises a camera 452 and an optical coherence imager 453, and may further comprise any other imaging apparatus. The optical coherence imager 453 transmits laser data to each of the photoacoustic imager 455 and the confocal scanning microscope 454 and images the tissue 43 of interest in cooperation with the camera 452, image data collected is sent to the console 46.

FIG. 4 shows a structural schematic diagram of a laser diagnosis and treatment system in an embodiment of this disclosure. As shown in FIG. 4, an embodiment of this disclosure further provides a laser diagnosis and treatment system. The laser diagnosis and treatment system comprises the implant 41 in various embodiments of this disclosure and a laser treatment apparatus 42 operated in cooperation with the implant 41 ingested to an organism. The implant 41 is ingested a tissue 43 of interest in an organism. An external light source module 44 in the laser treatment apparatus 42 emits visible and near infrared light from the outside of an organism to excite the implant 41 in a state of binding to a tissue 43 of interest. The implant 41 is excited to form a laser source in the organism. The laser source emits laser acting on the tissue 43 of interest to treat the tissue 43 of interest. The imaging module 45 performs local radiography or three-dimensional imaging on the tissue 43 of interest and sends image data to the console 46. The console 46 receives and processes image data so as to guide the process of treatment and to assess the therapeutic effect. Here, the energy density of the visible and near infrared light emitted from the outside of the organism by the external light source module 44 is lower than those of existing medical laser sources, so that severe damages to normal tissues of the organism may be prevented, and the safety of this laser diagnosis and treatment system is relatively high.

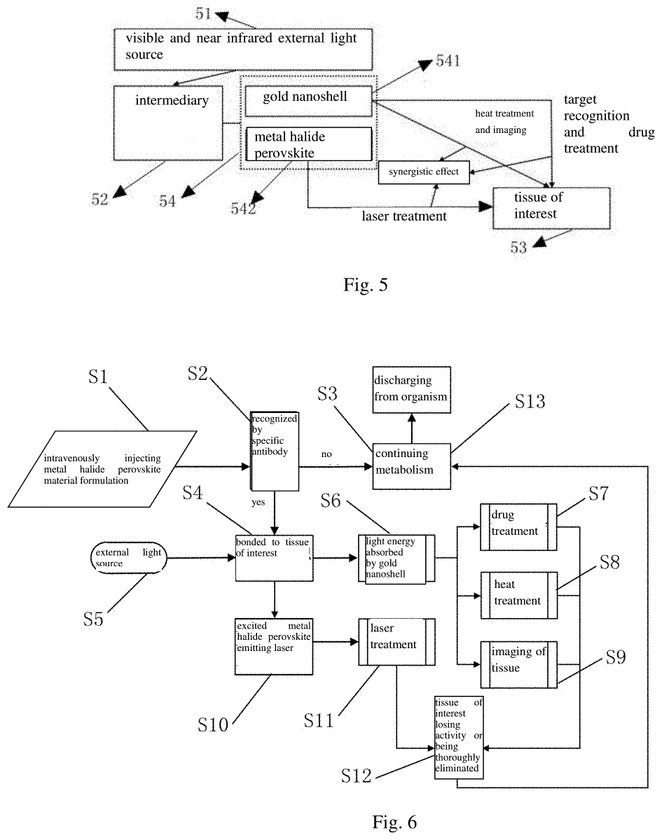

Particularly, as shown in FIG. 5, visible and near infrared light emitted from an externally applied visible-near infrared light source 51 acts on a laser source 54 in a state of being bonded to a tissue 53 of interest through an intermediary 52. A gold nanoshell 541 in an implant 54 recognizes the tissue 53 of interest, while the gold nanoshell 541 absorbs energy of visible and near infrared light and then releases a drug supported thereon so as to perform drug treatment on the tissue 53 of interest. Further, the gold nanoshell 541 absorbs energy of visible and near infrared light and then releases thermal energy on the tissue 53 of interest so as to perform heat treatment on the tissue 53 of interest. Moreover, the gold nanoshell 541 serves as a contrast agent after absorbing energy of visible and near infrared light, as so as to perform imaging on the tissue 53 of interest. A metal halide perovskite 542 in the implant 54 is excited by visible and near infrared light to form an in vivo laser source and then emits laser having a specific wavelength acting on the tissue 53 of interest so as to perform laser treatment on the tissue 53 of interest. The laser emitted from the metal halide perovskite 542 in the laser diagnosis and treatment system has good penetrability for the tissue 53 of interest and does not damage other tissues surrounding the tissue 53 of interest, and may synergistically act on the tissue 53 of interest in cooperation with the gold nanoshell 541, so as to achieve various treatments on the tissue 53 of interest. This system has high safety and relatively strong generality.

Furthermore, FIG. 6 is a flow chart of laser treatment in an embodiment of this disclosure. As shown in FIG. 6, an implant formulation is intravenously injected to an organism (S1). A tissue of interest is recognized by a specific antibody in the implant (S2). If the tissue of interest is not recognized by the specific antibody, the implant subsequently participates in metabolism of a human body (S3) so as to be discharged from the body. If the tissue of interest is recognized by the specific antibody, the implant binds to the tissue of interest (S4). An external light source then emits visible and near infrared light acting on the tissue of interest (S5). A gold nanoshell in the implant absorbs light energy (S6). A drug supported thereon is released to the tissue of interest (S7). The tissue of interest is subjected to heat treatment (S8). The gold nanoshell is used as a contrast agent to perform imaging on the tissue of interest in cooperation with an external image collection apparatus (S9). A metal halide perovskite in the implant is excited by the visible and near infrared light emitted from the external light source to form an in vivo laser source at the same time (S10). In situ laser acting on the tissue of interest is emitted so as to perform laser treatment on the tissue of interest (S11). The tissue of interest loses activity or is thoroughly eliminated after synergistic treatment of the gold nanoshell and the metal halide perovskite (S12). And the implant subsequently participates in metabolism of a human body (S13) so as to be discharged from the body.

In some examples, there is provided a method of forming a laser source by using a metal halide perovskite, comprising: exciting the metal halide perovskite in a state of binding to a tissue of interest by visible and near infrared light applied from the outside of an organism to form an in vivo laser source.

In some embodiments, the metal halide perovskite employs at least one structure of a nanosheet, a nanowire, and a quantum dot so as to form a microscale and/or nanoscale in vivo laser source under excitation.

In some embodiments, the metal halide perovskite comprises a metal halide perovskite 141 having a two-dimensional structure, and has a general chemical formula of AMX₃, wherein A represents a monovalent cation, M represents a bivalent metal ion, and X represents a halogen ion.

In some embodiments, the method comprises: extending a wavelength range of laser emitted a metal halide perovskite by at least one of adjusting a stoichiometric ratio of the metal halide perovskite, replacing a halogen or mixed halogens, and replacing an organic element with an inorganic element.

In some embodiments, the method comprises: combining a metal halide perovskite with a gold nanoshell to obtain the metal halide perovskite.

In some embodiments, the method further comprises: allowing the gold nanoshell to achieve the coupling to a specific antibody by surface modification.

In some embodiments, the method further comprises: allowing the gold nanoshell to achieve the coupling to and/or the supporting of a therapeutic targeted drug by surface modification.

In some embodiments, the coupling is achieved by ligand modification.

In some embodiments, the internal cavity and/or the surface of the gold nanoshell are configured to support a drug.

In some embodiments, the method further comprises: controlling the radiation of the visible and near infrared light applied from the outside of the organism to control the release of the drug supported.

In some examples, there is provided a laser treatment apparatus. The laser treatment apparatus is operated in cooperation with the metal halide perovskite ingested to an organism by using the method in various embodiments according to this disclosure, and comprises: an external light source module, configured to apply visible and near infrared light from the outside of an organism.

In some embodiments, the visible and near infrared light applied by the external light source module has an energy density in orders of magnitude of 10⁻⁷J cm⁻² to 10⁻⁶J cm⁻².

In some embodiments, the laser treatment apparatus further comprises: an imaging module, configured to perform imaging on the tissue of interest under the action of the in vivo laser source; a console, configured to receive and process image data from the imaging module.

In some embodiments, the external light source module comprises a light source emitter, an adapter, a first beam splitter, a power meter, and a microscope, wherein the adapter is used for adapting the first beam splitter, the visible and near infrared light emitted from the light source emitter is transmitted to the first beam splitter and is then transmitted through the first beam splitter to each of the tissue of interest, the power meter, and the microscope.

In some embodiments, the imaging module comprises a second beam splitter, a camera, an optical coherence imager, a confocal scanning microscope, and a photoacoustic imager, wherein the laser emitted the in vivo laser source is transmitted to the second beam splitter and is then transmitted through the second beam splitter to each of the camera and the optical coherence imager.

In some examples, there is provided a laser diagnosis and treatment system, comprising: a metal halide perovskite, which may be ingested to an organism; and the laser treatment apparatus in various embodiments according to this disclosure, which is configured to be operated in cooperation with the metal halide perovskite ingested to the organism so as to excite the metal halide perovskite to form a laser source in the organism.

Compared to the prior art, the advantageous effects of this disclosure are as follows.

1. The in vivo laser source formed by using a metal halide perovskite in this disclosure may act on target regions in a directed manner. The energy density of the visible and near infrared light, which is applied from the outside of an organism and is used for exciting a metal halide perovskite, is lower than those of existing medical laser sources. Therefore, irreversible damages to normal tissues of the organism may be prevented, and the safety is relatively high.

2. The laser diagnosis and treatment system in this disclosure may eliminate damages to normal tissues caused by externally applied laser in existing laser treatment means. Photothermal treatment, laser treatment, and drug treatment are integrated to achieve synergistic treatment and real-time imaging is performed on the tissue of interest at the same time so as to guide the process of treatment and to assess the therapeutic effect. The system has strong generality and relatively high safety.

The above description is intended to be illustrative, and not restrictive. For example, the examples described above (or one or more solutions thereof) may be used in combination with each other. For example, other embodiments may be used by those of ordinary skill in the art upon reading the description described above. Additionally, in the specific embodiments described above, various features may be grouped together to simplify this disclosure. This should not be interpreted as intending that an unclaimed disclosed feature is essential to any claim. Rather, the subject matter of this disclosure may be less than all features of a particular disclosed embodiment. Thus, the following claims are hereby incorporated as examples or embodiments into the specific embodiments, with each claim independently used as a separate embodiment, and it is contemplated that these embodiments may be combined with each other in various combinations or permutations. The scope of this disclosure should be determined with reference to the appended claims, along with the full scope of equivalents to which such claims are entitled.

The embodiments described above are merely exemplary embodiments of this disclosure and are not intended to limit this disclosure. The protection scope of this disclosure is determined by the appended claims. various modifications or equivalent replacements may be made to this disclosure by those skilled in the art within the spirit and the protection scope of this disclosure. These modifications or equivalent replacements should be also deemed to fall within the protection scope of this disclosure.

What is claimed is:

1. An implant for laser diagnosis and treatment, wherein the implant comprises:
a metal halide perovskite, wherein the metal halide perovskite is in a form of a nanosheet, a nanowire, or a quantum dot;
a gold nanoshell coupled to the metal halide perovskite; and
an antibody, which is bondable to a biological tissue, on an outer surface of the gold nanoshell.

2. The implant of claim 1, wherein:
the metal halide perovskite is a metal halide perovskite having a two-dimensional structure.

3. The implant of claim 1, wherein:
the metal halide perovskite has a general chemical formula of AMX₃, wherein A represents a monovalent cation, M represents a bivalent metal ion, and X represents a halogen ion.

4. The implant of claim 3, wherein:
A is selected from CH₃NH₃⁺, Cs⁺, and Rb⁺, M is selected from Pb²⁺ and Sn²⁺, and X is selected from Cl⁻, Br⁻, and I⁻.

5. The implant of claim 1, wherein:
the antibody is a specific antibody.

6. The implant of claim 5, wherein:
the specific antibody comprises a protein, a polypeptide, DNA, or a drug.

7. The implant of claim 1, wherein:
the surface of the gold nanoshell is coupled to the antibody through a ligand.

8. The implant of claim 1, wherein:
a therapeutic targeted drug is supported on an internal cavity or a surface of the gold nanoshell.

9. A laser diagnosis and treatment device, wherein the laser diagnosis and treatment device comprises:
a light source module configured to emit visible light or infrared light for exciting the implant of claim 1 in an organism to emit a laser.

10. The diagnosis and treatment device of claim 9, wherein:
the light source module comprises a light source emitter, a first beam splitter, a power meter, and a microscope, wherein the first beam splitter splits light emitted from the light source emitter to the implant, the power meter, and the microscope.

11. The diagnosis and treatment device of claim 10, wherein the diagnosis and treatment device further comprises:
an imaging module configured to image the laser emitted from the implant under excitation of the light source module.

12. The diagnosis and treatment device of claim 11, wherein:
the imaging module comprises a second beam splitter and a plurality of imaging apparatuses, wherein the second beam splitter splits the laser to the plurality of imaging apparatuses.

13. The diagnosis and treatment device of claim 12, wherein:
the plurality of imaging apparatuses comprise a camera and an optical coherence imager.

14. A method of emitting a laser by using the implant of claim 1, wherein the method comprises:
allowing the implant to be bonded to a biological tissue in an organism by the antibody, and
emitting visible or near infrared light by using an external light source outside the organism to excite the implant to emit the laser.

15. The method of claim 14, wherein:
the visible or near infrared light has an energy density between 1×10⁻⁷ J cm⁻² to 1×10⁻⁵ J cm⁻².

16. The method of claim 15, wherein:
the biological tissue is subjected to laser treatment or laser imaging by using the laser.
